(19) United States
(12) Patent Application Publication
(10) Pub. No.: US 2020/0170841 A1
(43) Pub. Date: Jun. 4, 2020

(54) DRESSINGS FOR FILTERING WOUND FLUIDS

(71) Applicants: KCI USA, INC., San Antonio, TX (US); SYSTAGENIX WOUND MANAGEMENT, LIMITED, West Sussex (GB)

(72) Inventors: Alexander WAITE, Keighley (GB); Craig DELURY, Gargrave (GB); Sylvia LINDSAY-WATT, Gargrave (GB)

(21) Appl. No.: 16/613,623

(22) PCT Filed: May 17, 2018
(86) PCT No.: PCT/US2018/033111

§ 371 Date: Nov. 14, 2019

Related U.S. Application Data
(60) Provisional application No. 62/508,918, filed on May 19, 2017.

(51) Int. Cl. A61F 13/00; A61M 1/00; A61L 15/28; A61L 15/32; A61L 15/24; A61L 15/44; A61K 33/38; A61L 15/42

(52) U.S. Cl. CPC A61F 13/00029; A61L 2300/404; A61F 13/00012; A61F 13/00017; A61F 13/00068; A61F 13/00063; A61L 15/28; A61L 15/325; A61L 15/24; A61L 15/44; A61K 33/38; A61L 15/425; A61F 2013/00634; A61M 2205/7536; A61L 2300/104; A61M 1/0088

(57) ABSTRACT

Disclosed herein are dressings capable of filtering, capturing, and/or releasing select biological molecules from wound exudate. In one embodiment, the dressing may include a permeable, non-adherent envelope. The permeable, non-adherent envelope may enclose a composition comprising milled hydrophobic foam.

### CROSS-REFERENCE TO RELATED APPLICATIONS

This application claims the benefit of priority to U.S. Provisional Application No. 62/508,918, filed on May 19, 2017, which is incorporated herein by reference in its entirety.

### BACKGROUND

A wide variety of materials and devices, generally characterized as “dressings,” are generally known in the art for use in treating a wound or other disruption of tissue. Such wounds may be the result of trauma, surgery, or disease, and may affect skin or other tissues. In general, dressings may control bleeding, absorb wound exudate, ease pain, assist in debriding the wound, protect wound tissue from infection, or otherwise promote healing and protect the wound from further damage.

Some dressings may protect tissue from, or even assist in the treatment of, infections associated with wounds. Infections can retard wound healing and, if untreated, can result in tissue loss, systemic infections, septic shock, and death. A variety of dressings are known in the art. Nevertheless, there remains a need for improved dressings having one or more characteristics such as improved antimicrobial efficacy, improved wound healing, improved absorption of blood and wound exudate, improved wound protection, reduced cost, and greater ease of use.

### BRIEF SUMMARY

Disclosed herein are dressings capable of filtering, capturing, and/or releasing select biological molecules from wound exudate. In some embodiments, the dressing may be configured to be positioned adjacent to a tissue site. The dressing may comprise a permeable, non-adherent envelope. The permeable, non-adherent envelope may enclose a composition comprising open-cell hydrophobic filter pieces.

Further are embodiments of a dressing, for example, for providing negative-pressure therapy to a tissue site. The system may comprise a dressing, which may be positioned adjacent to the tissue site. The dressing may comprise a permeable, non-adherent envelope. The permeable, non-adherent envelope may enclose a composition comprising open-cell hydrophobic filter pieces. The system may also comprise a secondary layer positioned adjacent to the dressing.

Further still are embodiments of a method for providing negative-pressure therapy to a tissue site. The method may comprise positioning a dressing adjacent to the tissue site. The dressing may comprise a dressing, a secondary layer positioned over the dressing, and a sealing member positioned adjacent the secondary layer. The dressing may comprise a permeable, non-adherent envelope. The permeable, non-adherent envelope may enclose a composition comprising open-cell hydrophobic filter pieces. The method may also comprise sealing the dressing to tissue surrounding the tissue site to form a sealed space. The method may also comprise fluidly coupling a negative-pressure source to the sealed space. The method may also comprise operating the negative-pressure source to generate a negative pressure in the sealed space.

Further still are embodiments of a method for enhanced wound healing. The method may comprise capturing at least some of a patient's growth factors or enzymes released into a dressing. The dressing may comprise a permeable, non-adherent envelope enclosing a composition comprising open-cell hydrophobic filter pieces. The method may also comprise releasing at least some of the captured growth factors from the dressing to the patient's wound.

Objectives, advantages, and illustrative modes of making and using the claimed subject matter may be understood best by reference to the accompanying drawings in conjunction with the following detailed description of illustrative embodiments.

### BRIEF DESCRIPTION OF THE DRAWINGS

FIG. 1 is a perspective, cross-sectional view of a dressing.

FIG. 2A is a perspective, cross-sectional view of the dressing of FIG. 1.

FIG. 2B is a cross-sectional view of the dressing of FIG. 1.

FIG. 3 is simplified schematic diagram of an example embodiment of a negative-pressure therapy system including a dressing.

FIG. 4 is a graph illustrating the results of an experiment demonstrating the capability of a dressing to bind growth factor.

FIG. 5 is a graph illustrating the results of an experiment demonstrating the capability of a dressing to release bound growth factor.

FIG. 6 is a graph illustrating the results of an experiment demonstrating the capability of the dressing to modulate protease activity.

### DESCRIPTION OF EXAMPLE EMBODIMENTS

The following description of example embodiments provides information that enables a person skilled in the art to make and use the subject matter set forth in the appended claims, but may omit certain details already well-known in the art. The following detailed description is, therefore, to be taken as illustrative and not limiting.

The example embodiments may also be described with reference to spatial relationships between various elements or to the spatial orientation of various elements depicted in the attached drawings. In general, such relationships or orientation assume a frame of reference consistent with or relative to a patient in a position to receive treatment. However, as should be recognized by those skilled in the art, this frame of reference is merely a descriptive expedient rather than a strict prescription.

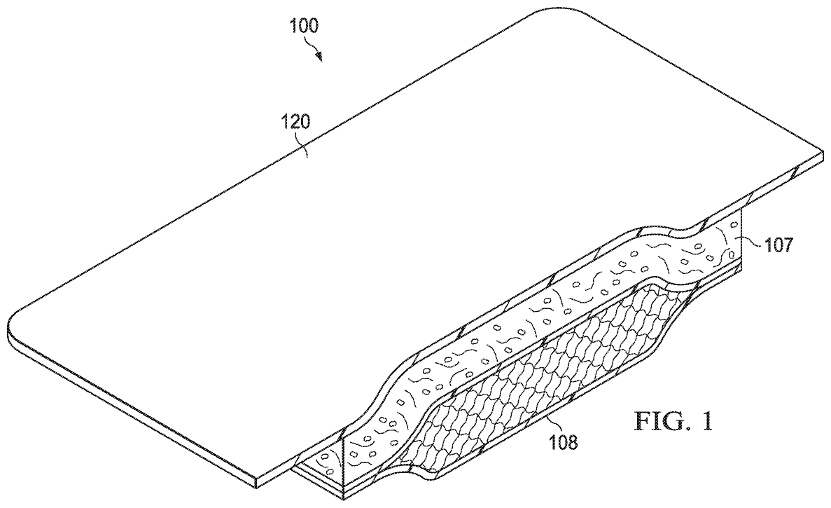

In some embodiments are dressings and therapy systems including the dressings. Also, in some embodiments are methods related to the dressings and therapy systems. For example, FIG. 1 illustrates an embodiment of a dressing 100. Generally, the dressing 100 may be configured to provide therapy to a tissue site in accordance with the disclosure of this specification.

“Tissue site” may broadly refer to a wound, defect, or other treatment target located on or within tissue, including but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, and grafts, for example. The term “tissue site” may also refer to areas of any tissue that are not necessarily wounded or defective, but are instead areas in which it may be desirable to add or promote the growth of additional tissue.

Dressing

In various embodiments, the dressing 100 may be generally configured to be in contact with the tissue site. For example, the dressing 100 may be in contact with a portion of the tissue site, substantially all of the tissue site, or the tissue site in its entirety. If the tissue site is a wound, for example, the dressing 100 may partially or completely fill the wound, or may be placed over (e.g., superior to) the wound. In various embodiments, the dressing 100 may take many forms, and may have many sizes, shapes, or thicknesses depending on a variety of factors, such as the type of treatment being implemented or the nature and size of a tissue site. For example, the size and shape of the dressing 100 may be adapted to the contours of deep and irregular shaped tissue sites and/or may be configured so as to be adaptable to a given shape or contour. Moreover, in some embodiments, any or all of the surfaces of the dressing 100 may comprise projections or an uneven, coarse, or jagged profile that can, for example, induce strains and stresses on a tissue site, for example, which may be effective to promote granulation at the tissue site.

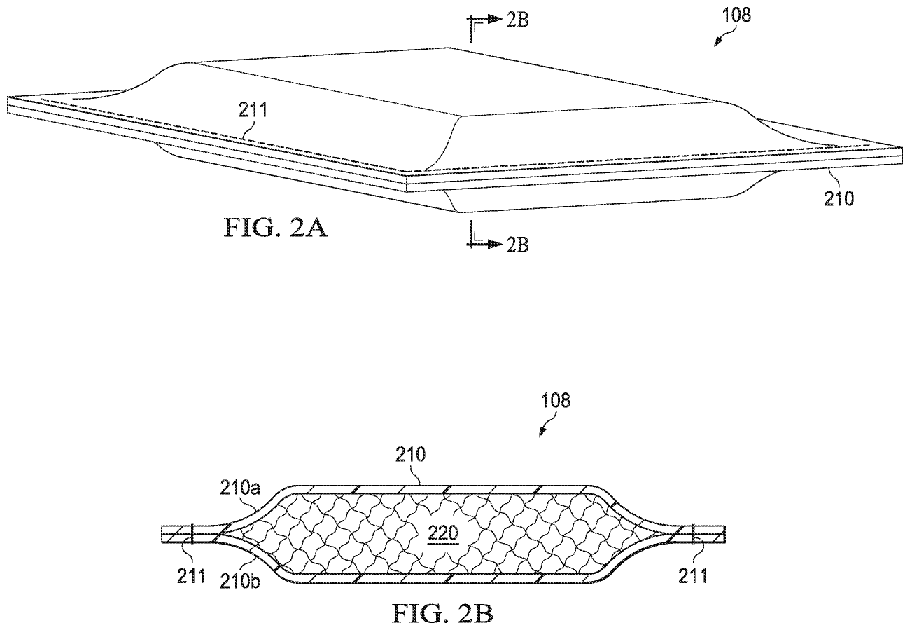

Referring to FIG. 2A, a perspective view of the dressing 100 of FIG. 1 is shown and, referring to FIG. 2B, a cross-sectional view of the dressing 100 is shown. In the embodiment of FIGS. 2A and 2B, the dressing 100 generally comprises an envelope 210 enclosing a composition of filter pieces 220.

Envelope

In some embodiments, the envelope 210 may be generally configured to enclose the composition of filter pieces 220, for example, to retain the composition of filter pieces 220 within a given, particular volume, which may have a given, particular shape. For example, in various embodiments the envelope 210 may be configured such that, when enclosing the composition of filter pieces 220, the resultant dressing 100 has a desired size and shape. In various embodiments, the envelope 210 may be characterized as a sack, a packet, a pouch, a tube, a cylinder, a box, a sphere or ball, or any suitable shape as may be suitably employed in a particular therapy. Additionally, in some embodiments the envelope 210 may comprise a plurality of volumes or compartments, each configured to enclose a quantity of the composition of filter pieces 220. For example, while the embodiment of FIGS. 2A and 2B illustrate the envelope as a generally rectangular pouch or packet, other configurations may be suitable.

In various embodiments, the envelope 210 may comprise or be formed from one or more sheets. The one or more sheets used to form the envelope 210 may have any suitable size and shape, for example, to yield an envelope having a particular or desired size or shape. For example, in various embodiments, the sheets may be square, rectangular, circular, oval, or oblong. In the embodiment of FIGS. 2A and 2B, the envelope 210 may comprise a first sheet, such as a top sheet 210a, and a second sheet, such as a bottom sheet 210b. In some embodiments, the top sheet 210a and the bottom sheet 210b may be coupled at a joint 211 extending about the periphery of the top sheet 210a and the bottom sheet 210b, for example, thereby defining a space within which the composition of filter pieces 220 may be enclosed. Additionally or alternatively, in some embodiments, an envelope like envelope 210 may further comprise one or more sheets joined to the top sheet 210a and the bottom sheet 210b. For example, one or more sheets can be joined to form sides or walls between the top sheet 210a and the bottom sheet 210b to increase the thickness of the envelope 210. In various embodiments, a joint between any two or more sheets, such as the joint 211 between the top sheet 210a and the bottom sheet 210b may take any suitable form. For example, in various embodiments, the joint 211 may comprise an adhesive (e.g., a medically-acceptable adhesive), stitching, a double-sided tape, or the like. In some embodiments, the envelope 210 may be formed from a single, folded sheet.

In various embodiments, the envelope 210 may comprise any suitable material and configuration. For example, in various embodiments, one or more sheets from which the envelope 210 may be formed may be a fibrous substrate, such as a woven material or a non-woven material, a film, or combinations thereof. The envelope 210 or one or more sheets from which the envelope 210 is formed may have a suitable thickness, for example, in the range of from about 100 μm to about 1,000 μm.

In some embodiments, the envelope 210 or one or more sheets from which the envelope 210 is formed may be formed from a suitable material. In some embodiments, the envelope 210 or one or more sheets from which the envelope 210 is formed may be characterized with respect to a tissue site as non-adherent, as having one or more non-adherent surfaces, or as being weakly adherent. For example, in various embodiments the envelope 210 or one or more sheets from which the envelope 210 is formed may comprise a normally flexible thermoplastic material, suitable examples of which include, but are not limited to, acrylics, acrylates, thermoplastic elastomers (for example, nylon, styrene ethylene butene styrene (SEBS) and other block copolymers), polyether block polyamide (PEBAX), silicone elastomers, poly caprolactam, poly lactic acid, and polyolefins, such as polyethylene and polypropylene. Examples of nylons suitable for use in forming the sheet and/or the envelope are available from Asahi Kasei Plastics in Fowlerville, Mich. as the Thermylon or Leona product lines.

In some embodiments, at least a portion of the envelope 210 or one or more sheets from which the envelope 210 is formed may be configured so as to be permeable to various wound fluids, for example, wound exudate or blood. For example, in some embodiments, the envelope 210 or one or more sheets from which the envelope 210 is formed may be characterized as porous, as perforated, or the like. For example, the envelope 210 or one or more sheets from which the envelope 210 is formed may comprise a plurality of pores extending there-through so as to allow communication through the envelope 210 or one or more sheets from which the envelope 210 is formed. In such embodiments, the plurality of pores may have an average pore size in the range of from about 200 μm to about 3,000 μm. Also in some embodiments, the plurality of pores may be present in the envelope 210 or one or more sheets from which the envelope 210 is formed at a pore density in the range of from about 2 pores/cm² to about 1,000 pores/cm².

Composition

In some embodiments, the composition of filter pieces 220 generally comprises a plurality of pieces of a suitable material. In some embodiments, the filter pieces 220 may be referred to as a “sized material,” for example, a material that has been “sized” to form the plurality of pieces of the material. As used in this context, a material that is characterized as having been “sized” may refer broadly to any material that may have been subjected to a process by which relatively small pieces result from processing of relatively large pieces of the material. Likewise, “sizing” may refer broadly to any process by which relatively small pieces result from such processing. Generally, examples of processes by which a material may be sized will be appreciated by one of skill in the art upon viewing this disclosure, suitable examples of which, include, but are not limited to, milling, pulverizing, shredding, tearing, cutting, chopping, grinding, macerating, grating, mincing, crumbling, or otherwise degrading, for example, by a mechanical process, a chemical process, or combinations thereof. Additionally or alternatively, in some embodiments a sized material may refer to pieces that may have one or more parameters substantially similar to those pieces resulting from such mechanical processes, chemical processes, or combinations thereof, regardless of the process by which the pieces are produced.

The filter pieces may have any suitable size and shape and may vary according to needs of a prescribed therapy. For example, in various embodiments the composition of filter pieces 220 may comprise a plurality of filter pieces of substantially the same shape (for example, a substantially homogenous mixture comprising filter pieces having substantially similar shapes), alternatively, the composition may comprise filter pieces of differing shapes (for example, a substantially heterogeneous mixture comprising filter pieces having various proportions of differently-shaped pieces), additionally or alternatively, the composition may comprise filter pieces of random shapes.

In some embodiments, the filter pieces 220 may be characterized on the basis of one or more of the dimensions of the filter pieces, for example, on the basis of an average of one or more of those dimensions and/or on the basis of a size distribution of one or more of those dimensions. Generally, a filter piece may have three quantifiable dimensions (e.g., x, y, and z directions and/or height, width, and depth). For example, the filter pieces may be characterized on the basis of a “major dimension” and a “minor dimension.” In this context, the “major dimension may refer to the largest relative dimension from among height, width, and depth in a given orientation and, likewise, the “minor dimension” may refer to the smallest relative dimension from among height, width, and depth in the same orientation. In various embodiments, the composition of filter pieces 220 may be characterized as having an average major dimension in the range of from about 0.5 mm to about 3.0 mm and an average minor dimension in the range of from about 0.5 to about 3.0 mm. Additionally or alternatively, in various embodiments, the composition of filter pieces 220 (e.g., a sized material) may be characterized as having a size distribution such that at least about 80% by weight of the filter pieces have a major dimension in the range of from about 1.0 mm to about 2.0 mm and a minor dimension in the range of from about 1.0 to about 2.0 mm, or more particularly, such that at least about 90% by weight of the filter pieces have a major dimension in the range of from about 1.0 mm to about 2.0 mm and a minor dimension in the range of from about 1.0 to about 2.0 mm, or more particularly, such that at least about 95% by weight of the filter pieces have a major dimension in the range of from about 1.0 mm to about 2.0 mm and a minor dimension in the range of from about 1.0 to about 2.0 mm.

In some embodiments, the composition of the filter pieces 220 may be configured such that at least some biological molecules such as growth factors or enzymes released proximate to the dressing 100, for example, from a patient receiving therapy, may be captured by the filter pieces 220. Additionally or alternatively, in some embodiments, the composition of filter pieces 220 may be configured to release at least some of the captured growth factors or enzymes if rinsed with a suitable solution, such as saline, for example. For example, and not intending to be bound by theory, one or more parameters associated with the composition of filter pieces 220 may be configured to allow growth factors or enzymes to be captured by the filter pieces 220. Also for example and not intending to be bound by theory, one or more parameters associated with the composition of filter pieces 220 may be configured to allow captured growth factors or enzymes to be released from the filter pieces 220 if rinsed with a suitable solution.

For example, in some embodiments, the material from which the filter pieces 220 are formed may have a porosity of from about 20 pores per inch to about 120 pores per inch. Also, in some embodiments, the material may have an average pore size in a range of from about 400 to about 600 microns. The number of pores and the average pore size of the material may vary according to needs of a prescribed therapy. In some embodiments, the material may be characterized as a hydrophobic, open-cell, reticulated foam. For example, in some embodiments, the material may be a hydrophobic, open-cell, reticulated polyurethane foam. Not intending to be bound by theory, the hydrophobic characteristics may prevent the material from directly absorbing fluid, such as blood or wound exudate (e.g., from the tissue site), but may allow the fluid to pass, for example, through the reticulated, open cells. Examples of suitable open-cell, reticulated polyurethane materials include, but are not limited to, a GranuFoam® dressing or of a VeraFlo® dressing, both available from Kinetic Concepts, Inc. of San Antonio, Tex.

In some embodiments, the composition of filter pieces 220 may be enclosed within the envelope 210 at any suitable rate or packing ratio. For example, in some embodiments, the composition of filter pieces 220 within the envelope may be characterized as tightly-packed, for example, such that the volume of the composition (when free or uncompressed) enclosed by the envelope is greater than the volume enclosed by the envelope. Alternatively, in some embodiments, the composition of filter pieces 220 within the envelope 210 may be characterized as loosely-packed, for example, such that the volume of the composition (when free or uncompressed) enclosed by the envelope 210 is less than the volume enclosed by the envelope 210. Additionally or alternatively, in some embodiments, the composition of filter pieces 220 may be enclosed within the envelope 210 such that the composition of filter pieces 220 may be characterized as being compressed such that composition of filter pieces 220 undergoes an increase in density of from about 1 mg/cm³ when free to about 100 mg/cm³ when packed within the envelope 210. For example, in some embodiments, an increase in density of from about 5 mg/cm³ to about 50 mg/cm³, on the basis of the volume of the envelope 210, of the composition of filter pieces 220 may be enclosed within the envelope 210.

In some embodiments, the composition of filter pieces 220 and/or the filter pieces 220 thereof may further comprise one or more active materials, for example, which may aid in wound healing. In some embodiments, the active materials may include non-steroidal anti-inflammatory drugs, steroids, antimicrobial agents such as antibiotics and antiseptics, enzymatic modulators (e.g., protease modulators), and combinations thereof. If present, active materials may be present in “safe and effective” amounts. Such safe and effective amounts may be sufficient to have the desired effect (e.g., antimicrobial activity), without undue risk of adverse side effects (such as toxicity, irritation, or allergic response), commensurate with a reasonable benefit/risk ratio when used in the manner of this technology. The specific safe and effective amount of an active material may vary with the active and various other factors such as the physical form of the active, the type and quantity of other materials in the filter pieces 220 and/or the composition, the intended use, and the physical condition of the subject on whom the dressings are intended to be used. In general, such active materials may be present in an amount of from about 0.1% to about 10%, by weight of the composition of filter pieces 220.

In various embodiments, the active materials may be integral within the filter pieces 220, may be incorporated within the pores of the filter pieces 220, may be included within the composition of filter pieces 220, or combinations thereof. For example, in some embodiments, an active material may be included within a slurry or resin from which the material of the filter pieces 220 may be formed to make the active material integral with the resultant filter pieces 220.

Additionally or alternatively, an active material may be incorporated after the material has been formed. For example, an active material may be incorporated into at least a portion of the pores or other void-spaces of the material. The active material may be incorporated in any suitable form, including a particulate form such as a powder or fiber, for example. Additionally or alternatively, an active material may be included within the composition of filter pieces 220, for example, as a particulate, such that the resultant composition of filter pieces 220 comprises a mixture of the filter pieces 220 and the active material.

For example, in some embodiments, the filter pieces 220 and/or the composition of filter pieces 220 may comprise a collagen and/or an oxidized regenerated cellulose (ORC). For example, in various embodiments, collagen, ORC, or both may be included within the composition as a particulate. In such an embodiment, the composition may include collagen, ORC, or both at any level appropriate, for example, to modulate protease activity and/or to modify various characteristics of the filter pieces 220 and/or composition of filter pieces 220. The collagen may be obtained from any natural source and may be Type I, II or III collagen, or may also be chemically modified collagen, for example an atelocollagen obtained by removing the immunogenic telopeptides from natural collagen. The collagen may also comprise solubilized collagen or soluble collagen fragments having molecular weights in the range of from about 5,000 to about 100,000, more particularly, from about 5,000 to about 50,000, obtained, for example, by pepsin treatment of natural collagen. In various embodiments, the collagen may be obtained from bovine corium that has been rendered largely free of non-collagenous components. Such non-collagenous components include fat, non-collagenous proteins, polysaccharides and other carbohydrates, as described in U.S. Pat. No. 4,614,794, Easton et al., issued Sep. 30, 1986 and U.S. Pat. No. 4,320,201, Berg et al., issued Mar. 16, 1982, incorporated by reference herein.

Generally, oxidized cellulose may be produced by the oxidation of cellulose, for example with dinitrogen tetroxide. Not intending to be bound by theory, this process converts primary alcohol groups on the saccharide residues to carboxylic acid groups, forming uronic acid residues within the cellulose chain. The oxidation may not proceed with complete selectivity, and as a result hydroxyl groups on carbons 2 and 3 may be converted to the keto form. These ketone units introduce an alkali labile link, which at pH 7 or higher initiates the decomposition of the polymer via formation of a lactone and sugar ring cleavage. As a result, oxidized cellulose may be biodegradable and bioabsorbable under physiological conditions. In some embodiments, the oxidized cellulose may be ORC, for example, prepared by oxidation of a regenerated cellulose, such as rayon. ORC may be manufactured by the process described in U.S. Pat. No. 3,122,479, Smith, issued Feb. 24, 1964, incorporated herein by reference. ORC is available with varying degrees of oxidation and hence rates of degradation. The ORC may be, for example, in the form of solid fibers, a sheet, a sponge, or a fabric. In various embodiments, the ORC comprises insoluble fibers, including woven, non-woven, and knitted fabrics. In other embodiments, the ORC may be in the form of water-soluble low molecular weight fragments, for example, obtained by alkali hydrolysis of ORC.

Examples of antiseptics among those useful in the filter pieces 220 and/or the composition of filter pieces 220 include silver, chlorhexidine, povidone iodine, triclosan, sucralfate, quaternary ammonium salts and mixtures thereof. In some embodiments, the filter pieces 220 may comprise silver, which may be in metallic form, in ionic form (e.g., a silver salt), or both. For example, in some embodiments the silver may be present in ionic form, such as in a complex with an anionic polysaccharide in the composition. In some embodiments, the composition of filter pieces 220 comprises a complex of silver and ORC (a “Silver/ORC Complex”). As referred to herein, such a complex may be an intimate mixture (e.g., at the molecular scale), for example, with ionic or covalent bonding between the silver and the ORC. The Silver/ORC Complex may comprise a salt formed between the ORC and Ag⁺, but may also comprise silver clusters or colloidal silver metal, for example produced by exposure of the complex to light. The complex of an anionic polysaccharide and silver contained may be made by treating the ORC with a solution of a silver salt. In various embodiments, the silver salt may be the salt of silver with a weak acid. Silver/ORC complexes useful herein, and methods of producing such complexes, are described in U.S. Pat. No. 8,461,410, Cullen et al., issued Jun. 11, 2013, incorporated by reference herein. Similar processes are described in U.S. Pat. No. 5,134,229, Saferstein et al., issued Jul. 28, 1992, incorporated by reference herein.

An example of antibiotics among those useful in the filter pieces 220 and/or the composition of filter pieces 220 include poly(hexamethylene biguanide) (“PHMB”), which is also known as polyaminopropyl biguanid (“PAPB”) and polyhexanide, having the following general formula.

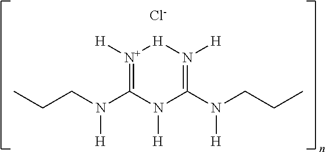

PHMB is a cationic broad spectrum antimicrobial agent. PHMB can be synthesized by a variety of methods, including polycondensation of sodium dicyanamide and hexamethylenediamine.

Cover

In some embodiments, the dressing 100 may comprise one or more additional layers. For example, in various embodiments, such additional layers may perform any of a variety of functions including, for example, adherence of the dressing to a tissue site or to surrounding tissues, increasing structural rigidity of the dressing, protection from moisture or other materials in the external environment, protection of a wound surface, delivery of one or more actives or other materials to the wound surface, or combinations thereof. In various embodiments, the additional layers may be conformable to a wound surface and/or to the surrounding tissues, for example, being capable of bending such that one or more of the additional layers may be in substantial contact with the wound and/or the surrounding tissues.

In some embodiments, the cover 120 may be positioned adjacent to the dressing 100, for example, so as to enclose the tissue site. The cover 120 may have a first surface and a second, opposite surface. The cover 120 may support one or more other layers of the dressing 100 on the first surface of the cover 120.

In various embodiments, the cover 120 may be generally configured to provide a bacterial barrier and protection from physical trauma. For example, the cover 120 may be constructed from a material that can reduce evaporative losses and provide a fluid seal between two components or two environments, such as between a therapeutic environment and a local external environment. The cover 120 may be, for example, an elastomeric film or membrane that can provide a seal adequate to maintain a negative pressure at a tissue site. In some embodiments, the cover 120 may have a high moisture-vapor transmission rate (MVTR), for some applications. For example, in such embodiments, the MVTR may be at least 300 g/m² per twenty-four hours. In some embodiments, the cover 120 may be formed from a suitable polymer. For example, the cover 120 may comprise a polymer drape, such as a polyurethane film, that may be permeable to water vapor but generally impermeable to liquid. For example, in such embodiments, such drapes have a thickness in the range of about 25 to about 50 microns.

In some embodiments, the cover 120 may be configured to be attached to an attachment surface, such as undamaged epidermis, a gasket, or another cover, for example, via an attachment device. In such embodiments, the attachment device may take any suitable form. For example, an attachment device may be a medically-acceptable, pressure-sensitive adhesive that extends about a periphery, a portion, or an entire sealing member. In some embodiments, for example, some or all of the cover 120 may be coated with an acrylic adhesive having a coating weight between 25-65 grams per square meter (g.s.m.). Thicker adhesives, or combinations of adhesives, may be applied in some embodiments, for example, to improve the seal and reduce leaks. Other example embodiments of an attachment device may include a double-sided tape, a paste, a hydrocolloid, a hydrogel, a silicone gel, or an organogel.

Secondary Layer

In various embodiments, the secondary layer 107 may be generally configured to collect fluid. For example, the secondary layer 107 may comprise or be configured as an absorbent layer. In some embodiments, the secondary layer 107 may be characterized as exhibiting absorbency. For example, the secondary layer 107 may exhibit an absorbency of at least 3 g saline/g, more particularly, at least 5 g saline/g, more particularly, from 8 to 20 g saline/g.

In some embodiments, the secondary layer 107 may comprise or may be formed from a suitable material, for example a foam, such as an open-cell foam or reticulated foam. In such embodiments, the average pore size of the foam may vary according to needs of a prescribed therapy, for example, a negative-pressure therapy. In some embodiments, the secondary layer 107 may be characterized as hydrophilic. For example, and not intending to be bound by theory, where the secondary layer 107 may be hydrophilic, the secondary layer 107 may be effective to absorb (e.g., wick) fluid away from the dressing 100. In such an embodiment, the wicking properties of the secondary layer 107 may be effective to draw fluid away from dressing 100 by capillary flow or other wicking mechanisms. An example of a hydrophilic foam is a polyvinyl alcohol, open-cell foam. Other hydrophilic foams may include those made from a polyether. Other foams that may exhibit hydrophilic characteristics include hydrophobic foams that have been treated or coated to provide hydrophilicity.

Negative-Pressure Therapy

Additionally, in some embodiments, the dressing 100 may be employed in a therapy in which a tissue site, for example, a wound, is treated with reduced pressure. Treatment of wounds or other tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and micro-deformation of tissue at a wound site. Together, these benefits may increase development of granulation tissue and reduce healing times.

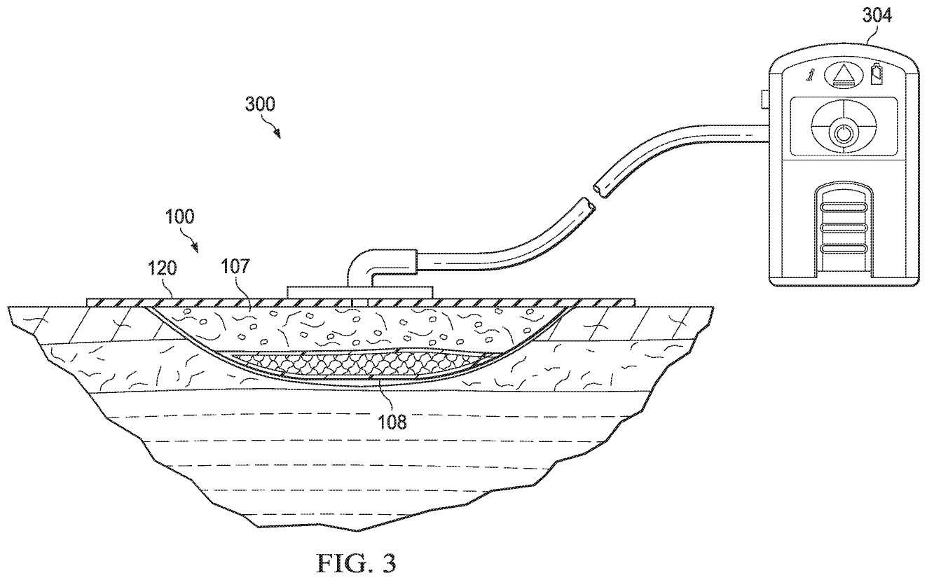

FIG. 3 is a simplified schematic that illustrates an example embodiment of a therapy system 300 for negative-pressure therapy. Generally, the therapy system 300 may be configured to provide negative-pressure to a tissue site. In various embodiments, the therapy system 300 generally includes a negative-pressure supply, and may include or be configured to be coupled to a distribution component. In general, a distribution component may refer to any complementary or ancillary component configured to be fluidly coupled to a negative-pressure supply in a fluid path between the negative-pressure supply and a tissue site. For example, in the embodiment of FIG. 3, the dressing 100 may be fluidly coupled to a negative-pressure source 304 such that negative pressure may be applied to a tissue site via the dressing 100. For example, the dressing 100, the secondary layer 107, or both may be generally configured to distribute negative pressure to collect fluid. In some embodiments, for example, the secondary layer 107, the dressing 100, or both may comprise or be configured as a manifold. A “manifold” in this context generally includes any composition or structure providing a plurality of pathways configured to collect or distribute fluid across a tissue site under pressure. For example, a manifold may be configured to receive negative pressure from the negative-pressure source 304 and to distribute negative pressure through multiple apertures (pores), which may have the effect of collecting fluid and drawing the fluid toward the negative-pressure source 304. For example, the secondary layer 107 may be configured to receive negative pressure from the negative-pressure source 304 and to distribute negative pressure to the dressing 100, which may have the effect of collecting fluid from the dressing 100 by drawing fluid from the tissue site through the dressing 100. Additionally or alternatively, the dressing 100 may be configured to receive negative pressure from the secondary layer 107 and/or from the negative-pressure source 304 and to distribute negative pressure to the tissue site, for example, which may have the effect of collecting fluid from the tissue site.

In some illustrative embodiments, the fluid pathways of a manifold may be interconnected to improve distribution or collection of fluids. For example, in some embodiments, a manifold may be a porous foam material having a plurality of interconnected cells or pores. For example, cellular foam, open-cell foam, reticulated foam, porous tissue collections, and other porous material such as gauze or felted mat generally include pores, edges, and/or walls adapted to form interconnected fluid pathways (e.g., channels). Liquids, gels, and other foams may also include or be cured to include apertures and fluid pathways. In some embodiments, a manifold may additionally or alternatively comprise projections or depressions that form interconnected fluid pathways.

The fluid mechanics associated with using a negative-pressure source to reduce pressure in another component or location, such as within a sealed therapeutic environment, can be mathematically complex. However, the basic principles of fluid mechanics applicable to negative-pressure therapy are generally well-known to those skilled in the art. The process of reducing pressure may be described generally and illustratively herein as “delivering,” “distributing,” or “generating” negative pressure, for example.

In general, a fluid, such as wound fluid (for example, wound exudates and other fluids), flow toward lower pressure along a fluid path. Thus, the term “downstream” typically implies something in a fluid path relatively closer to a source of negative pressure or further away from a source of positive pressure. Conversely, the term “upstream” implies something relatively further away from a source of negative pressure or closer to a source of positive pressure. This orientation is generally presumed for purposes of describing various features and components herein. However, the fluid path may also be reversed in some applications (such as by substituting a positive-pressure source for a negative-pressure source) and this descriptive convention should not be construed as a limiting convention.

As used herein, “negative pressure” is generally intended to refer to a pressure less than a local ambient pressure, such as the ambient pressure in a local environment external to a sealed therapeutic environment provided by the dressing 100. In many cases, the local ambient pressure may also be the atmospheric pressure proximate to or about a tissue site. Alternatively, the pressure may be less than a hydrostatic pressure associated with the tissue at the tissue site. While the amount and nature of negative pressure applied to a tissue site may vary according to therapeutic requirements, the pressure is generally a low vacuum, also commonly referred to as a rough vacuum, between −5 mm Hg (−667 Pa) and −500 mm Hg (−66.7 kPa). Common therapeutic ranges are between −75 mm Hg (−9.9 kPa) and −300 mm Hg (−39.9 kPa).

In various embodiments, a negative-pressure supply, such as the negative-pressure source 304, may be a reservoir of air at a negative pressure, or may be a manual or electrically-powered device that can reduce the pressure in a sealed volume, such as a vacuum pump, a suction pump, a wall suction port available at many healthcare facilities, or a micro-pump, for example. A negative-pressure supply may be housed within or used in conjunction with other components, such as sensors, processing units, alarm indicators, memory, databases, software, display devices, or user interfaces that further facilitate therapy. For example, in some embodiments, the negative-pressure source 304 may be combined with a controller and other components into a therapy unit. A negative-pressure supply may also have one or more supply ports configured to facilitate coupling and de-coupling of the negative-pressure supply to one or more distribution components.

In various embodiments, components may be fluidly coupled to each other to provide a path for transferring fluids (i.e., liquid and/or gas) between the components. For example, components may be fluidly coupled through a fluid conductor, such as a tube. As used herein, the term “fluid conductor” may broadly include a tube, pipe, hose, conduit, or other structure with one or more lumina adapted to convey a fluid between two ends thereof. Typically, a fluid conductor may be an elongated, cylindrical structure with some flexibility, but the geometry and rigidity may vary. In some embodiments, the negative-pressure source 304 may be operatively coupled to the dressing 100 via a dressing interface. For example, in the embodiment of FIG. 3, the dressing 100 may be coupled to the negative-pressure source 304 via a dressing interface such that the dressing 100 receives negative pressure from the negative-pressure source 304.

Methods

In some embodiments are methods for treating a tissue site such as a wound, for example, in the context of various therapies.

In some embodiments, a therapy method may comprise positioning the dressing 100 for treating a tissue site. For example, in operation, the dressing 100 may be positioned proximate to the tissue, such as a wound. The dressing 100 may be used with any of a variety of wounds, such as those occurring from trauma, surgery or disease. For example, such wounds may be chronic wounds, venous ulcers, decubitus ulcers or diabetic ulcers. For example, the dressing 100 may be placed within, over, on, or otherwise proximate to the tissue site. Additionally, in some embodiments, the cover 120 and secondary layer 107, may be placed and the cover 120 sealed to an attachment surface near the tissue site. For example, the cover 120 may be sealed to undamaged epidermis peripheral to a tissue site. In some embodiments, the dressing 100 may be positioned first and, after the dressing 100 has been positioned, the secondary layer 107 and cover 120 may be positioned. In some other embodiments, the dressing 100 may be preassembled, for example, such that the various layers of the dressing 100 are positioned with respect to each other prior to placement proximate the tissue site. Thus, the cover 120 can provide a sealed therapeutic environment including the secondary layer 107 and dressing 100 and proximate to a tissue site, substantially isolated from the external environment.

In some embodiments, the dressing 100 may be used in a negative-pressure therapy method. The negative-pressure therapy method may comprise positioning a dressing adjacent to the tissue site. For example, in operation, the dressing 100 may be placed within, over, on, or otherwise proximate to a tissue site. The cover 120 may be sealed to an attachment surface near the tissue site. In various embodiments, the various components of the dressing 100 may be positioned with respect to the tissue site sequentially or, alternatively, may be positioned with respect to each other and then positioned with respect to the tissue site.

The negative-pressure therapy method may further comprise sealing the dressing 100 to tissue surrounding the tissue site to form a sealed space. For example, the cover 120 may be sealed to undamaged epidermis peripheral to a tissue site such that the dressing 100 may provide a sealed therapeutic environment proximate to a tissue site, substantially isolated from the external environment.

The negative-pressure therapy method may further comprise fluidly coupling a negative-pressure source to the sealed space and operating the negative-pressure source to generate a negative pressure in the sealed space. For example, the negative-pressure source 304 may be coupled to the dressing 100 such that the negative-pressure source 304 may be used to reduce the pressure in the sealed space. For example, negative pressure applied across the tissue site, for example, via the secondary layer 107 and/or the dressing 100 in the sealed space can induce macrostrain and microstrain at the tissue site, as well as remove exudates and other fluids from the tissue site.

In some embodiments, the therapy method may further comprise capturing growth factors or enzymes via the dressing. In some embodiments, the absorbency of the secondary layer 107 may be effective to draw fluid through the dressing 100 so as to filter biological molecules from the fluid. For example, the secondary layer 107 may be an absorbent layer. The dressing 100 may filter growth factors and/or enzymes from the fluid, for example, by trapping the biological molecules within the pores of the filter pieces 220. Additionally or alternatively, in some embodiments, operating the negative-pressure source 304 may be effective to draw fluid from the sealed space through the dressing 100 so as to filter biological molecules such as growth factors and/or enzymes, which can trap the biological molecules within the pores of the filter pieces of the dressing 100.

In some embodiments, the therapy method may further comprise modulating enzyme activity, for example, protease activity, at the tissue site by capturing the enzymes proteases within the dressing 100 and removing the dressing 100 with the captured enzymes. Additionally, in some embodiments, the activity of the captured enzymes, such as proteases, may be modulated by the dressing 100. For example, in some embodiments, the dressing 100 may comprise an active material having protease-modulating activity, such as collagen and/or ORC, more particularly, collagen and ORC. For example, the presence of collagen and/or ORC, the collagen and/or ORC may be effective to modulate protease activity.

In some embodiments, the therapy method may further comprise delivering signaling molecules, particularly, growth factors, to the tissue site. For example, the captured the signaling molecules, particularly, growth factors, within the dressing 100 may be rinsed from the dressing 100, for example, with a suitable solution such as saline, and returned to the tissue site.

Advantages

The dressings and therapy systems may provide significant advantages, for example, when used in a therapy.

For example, in some embodiments, dressings and systems may be advantageously employed to recover growth factors and to deliver those growth factors to a tissue site. For example, when the dressings and systems are used in a therapy, wound fluid (e.g., wound exudate) may be drawn through the dressing 108, for example, via the absorptive action of the secondary layer and/or via the application of negative pressure. Not intending to be bound by theory, as the wound fluid is drawn through the dressing, various biological molecules present within the wound fluid, for example, growth factors such as platelet-derived growth factor (PDGF) (e.g., PDGF-BB), fibroblast growth factor (FGF), and epidermal growth factor (EGF), or combinations thereof, may become trapped within the dressing. For example, the various biological molecules may be trapped in the gaps between various filter pieces and/or by or within the pores of the filter pieces. In some embodiments, the dressing may be removed and replaced, and a physician or other caregiver may rinse the dressing and return at least a portion of the rinsate to the tissue site, which can deliver growth factors to the tissue site. For example, the dressing may be removed upon a particular duration or as otherwise necessitated in the course of a therapy, and may be rinsed with a sterile saline solution. More particularly, and not intending to be bound by theory, upon rinsing the dressing (after use in a therapy), at least a portion of the growth factors trapped by or within the dressing may be released from the dressing and be present within the resultant rinsate. As such, the rinsate may comprise a source of autologous growth factors that may be returned to the tissue site. Growth factors, such as platelet-derived growth factor (PDGF) (e.g., PDGF-BB), fibroblast growth factor (FGF), and epidermal growth factor (EGF), may be beneficial to wound healing, particularly, when insufficient amounts of such growth factors are present at a tissue site (e.g., at a wound). As such, in some embodiments, the capability to use the dressings and systems to recover growth factors (for example, by trapping by or within the dressing) and to deliver the recovered growth factors to the tissue site may be beneficial to wound healing, for example, by decreasing the duration of the wound healing process (e.g., wound chronicity).

Also for example, in some embodiments, the dressings and systems may be advantageously employed to modulate protease activity. For example, if the dressings and systems are used in a therapy, wound fluid (e.g., wound exudate) may be drawn through the dressing via the absorptive action of the secondary layer and/or via the application of negative pressure. Not intending to be bound by theory, as the wound fluid is drawn through the dressing, various biological molecules in the wound fluid, for example, enzymes such as proteases, may become trapped within the dressing. In some embodiments, the dressing may be removed and replaced, for example by a physician or other caregiver, thereby removing the enzymes (e.g., proteases, such as matrix metallopeptidase 9 (MMP-9)) from proximity with the tissue site. Enzymes, such as proteases, may be destructive or detrimental to wound healing, particularly, when an over-abundance of such proteases are present at a tissue site. As such, in some embodiments, the capability to use the dressings and systems to remove enzymes trapped by the dressing from proximity with the tissue site may be beneficial to wound healing, for example, by decreasing the duration of the wound healing process (e.g., wound chronicity). For example, using the dressings and systems to remove enzymes may be effective to reduce enzymatic activity at a tissue site, particularly, proteolytic activity such as by MMP-9, to about 35% relative to proteolytic activity at the tissue site that received no such therapy (e.g., a positive control), more particularly, to about 30%, more particularly, to about 20%, more particularly, to about 10%, more particularly, to about 5%, more particularly, to less than about 1%.

Additionally, in some embodiments where the dressing comprises an active material having protease-modulating activity, such as collagen and/or ORC, more particularly, collagen and ORC, the presence of the active material (e.g., collagen and/or ORC) may further reduce enzymatic activity, particularly, proteolytic activity, such as by MMP-9 or Human Neutrophil Elastase (HNE). For example, using the dressings and systems to remove the enzymes (e.g., proteases) may be effective to further reduce enzymatic activity, particularly, proteolytic activity such as by MMP-9, to about less than about 1%.

### EXAMPLES

One or more of the advantages associated with the dressings and various solutions may be further demonstrated by the following, non-limiting examples. These examples may demonstrate one or more features associated with some embodiments of the dressings and systems.

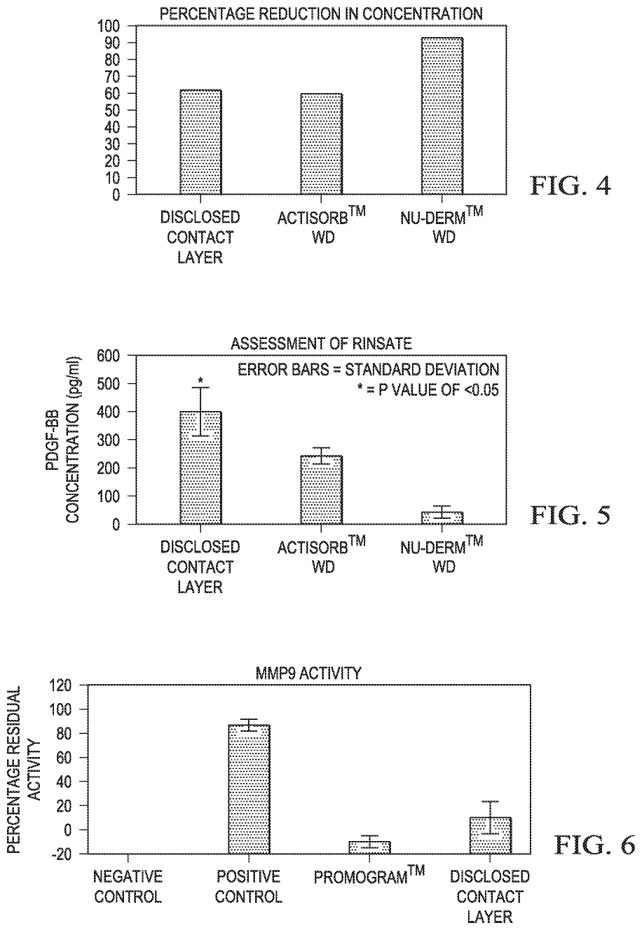

In Example 1, the capability of a dressing (for example, a dressing comprising a permeable, non-adherent envelope enclosing a composition comprising open-cell hydrophobic filter pieces, collagen, and ORC) to bind growth factor is demonstrated. In Example 1, a dressing was compared with other, commercially-available dressings, particularly, the Actisorb™ Wound Dressing (e.g., an activated charcoal dressing enclosed in a non-adherent nylon envelope) and the NU-DERM™ Alginate Wound Dressing (e.g., an alginate-containing non-adherent dressing), both available from Acelity, Inc. in San Antonio, Tex. Each of the dressing, the Actisorb™ Wound Dressing, and the NU-DERM™ Alginate Wound Dressing were incubated for 2 hours in a 2 ml solution of PDGF-BB (2,000 pg/ml) and BSA (20 pg/ml) as a diluent. The results are illustrated in FIG. 4.

As shown in FIG. 4, each of the dressing, the Actisorb™ Wound Dressing, and the NU-DERM™ Alginate Wound Dressing significantly reduced the concentration of PDGF-BB in the solution. Thus, Example 1 demonstrates the ability of the dressing to bind growth factor (particularly, PDGF and, more particularly, PDGF-BB).

Also, in Example 2, the capability of the dressing to release bound growth factor is demonstrated. In Example 2, following the incubation of the dressing, the Actisorb™ Wound Dressing, and the NU-DERM™ Alginate Wound Dressing, each was rinsed with 2 ml of BSA solution (20 pg/ml). The rinsate from each of these was assessed for PDGF-BB concentration. The results are illustrated in FIG. 5.

As shown in FIG. 5, the dressing was significantly more effective at releasing bound growth factor (particularly, PDGF and, more particularly, PDGF-BB), in comparison to the Actisorb™ Wound Dressing and the NU-DERM™ Alginate Wound Dressing.

In Example 3, the capability of the dressing to modulate protease activity is demonstrated. In Example 3, the dressing was incubated with a protease solution containing MMP9. Following incubation, aliquots of the MMP9 solution were combined with a substrate that, when cleaved by the MMP9 enzyme, would yield fluorescence. Fluorescence, therefore, can be used as a measure of enzymatic activity. The results are illustrated in FIG. 6.

As shown in FIG. 6, the dressing was effective to reduce protease activity (particularly, MMP9 activity) to about 30% of the protease activity of the positive control. The MMP9 solution was also exposed to Promogran™, a collagen and ORC dressing known yield a reduction in protease activity.

The description and specific examples, while indicating embodiments of the technology, are intended for purposes of illustration only and are not intended to limit the scope of the technology. Moreover, recitation of multiple embodiments having stated features is not intended to exclude other embodiments having additional features, or other embodiments incorporating different combinations of the stated features. Components may be also be combined or eliminated in various configurations for purposes of sale, manufacture, assembly, or use. Specific examples are provided for illustrative purposes of how to make and use the compositions and methods of this technology and, unless explicitly stated otherwise, are not intended to be a representation that given embodiments of this technology have, or have not, been made or tested. Equivalent changes, modifications and variations of some embodiments, materials, compositions and methods can be made within the scope of the present technology, with substantially similar results.

As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features. Moreover, descriptions of various alternatives using terms such as “or” do not require mutual exclusivity unless clearly required by the context, and the indefinite articles “a” or “an” do not limit the subject to a single instance unless clearly required by the context.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of” Thus, for any given embodiment reciting materials, components or process steps, the present technology also specifically includes embodiments consisting of, or consisting essentially of, such materials, components or processes excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

Disclosure of values and ranges of values for specific parameters (such as temperatures, molecular weights, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, and 3-9.

The term “about,” as used herein, is intended to refer to deviations in a numerical quantity that may result from various circumstances, for example, through measuring or handling procedures in the real world; through inadvertent error in such procedures; through differences in the manufacture, source, or purity of compositions or reagents; from computational or rounding procedures; and other deviations as will be apparent by those of skill in the art from the context of this disclosure. For example, the term “about” may refer to deviations that are greater or lesser than a stated value or range by 1/10 of the stated value(s), e.g., ±10%, as appropriate from the context of the disclosure. For instance, a concentration value of “about 30%” may refer to a concentration between 27% and 33%. Whether or not modified by the term “about,” quantitative values recited in the claims include equivalents to the recited values, for example, deviations from the numerical quantity, as would be recognized as equivalent by a person skilled in the art in view of this disclosure.

The appended claims set forth novel and inventive aspects of the subject matter disclosed and described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described herein may also be combined or replaced by alternative features serving the same, equivalent, or similar purpose without departing from the scope of the invention defined by the appended claims.

1. A dressing comprising:
a composition comprising hydrophobic filter pieces;
a permeable, non-adherent envelope enclosing the composition;
wherein the dressing is configured to capture biological molecules.

2. The dressing of claim 1, wherein the dressing is configured to release at least a portion of captured biological molecules.

3. The dressing of claim 2, wherein the dressing is configured to release at least a portion of captured biological molecules when the dressing is rinsed with a liquid.

4. The dressing of claim 2, wherein the captured biological molecules comprise signaling proteins and enzymes.

5. The dressing of claim 4, wherein the signaling proteins comprise growth factors.

6. The dressing of claim 4, wherein the enzymes comprise proteases.

7. (canceled)

8. The dressing of claim 1, wherein the composition further comprises collagen.

9. (canceled)

10. The dressing of claim 1, wherein the composition further comprises oxidized regenerated cellulose (ORC).

11. (canceled)

12. The dressing of claim 1, wherein the composition further comprises collagen and ORC.

13. (canceled)

14. The dressing of claim 1, wherein the hydrophobic filter pieces have a size distribution such that at least about 80% by weight of the filter pieces have a major dimension in the range of from about 1.0 mm to about 2.0 mm and a minor dimension in the range of from about 1.0 to about 2.0 mm.

15. The dressing of claim 1, wherein the hydrophobic filter pieces comprise a milled foam.

16. The dressing of one of claim 15, wherein the milled foam is made of polyurethane.

17. The dressing of claim 1, wherein the composition comprises an antimicrobial agent.

18. The dressing of claim 17, wherein the antimicrobial agent comprises silver.

19. The dressing of claim 1, wherein the permeable, non-adherent envelope comprises a nylon.

20. The dressing of claim 1, wherein the permeable, non-adherent envelope comprises at least a portion having a pore density in the range of from about 2 pores/cm² to about 1,000 pores/cm².

21. A dressing comprising:
a permeable, non-adherent envelope enclosing a composition comprising hydrophobic filter pieces; and
a secondary layer adjacent to the permeable, non-adherent envelope.

22. The dressing of claim 21, wherein the secondary layer is an absorbent layer configured to absorb wound exudate.

23. The dressing of claim 21, wherein the secondary layer is a backing layer configured to adhere the dressing to a user.

24. The dressing of claim 21, wherein the composition further comprises collagen.

25. (canceled)

26. The dressing of claim 24, wherein the composition further comprises oxidized regenerated cellulose (ORC).

27-29. (canceled)

30. The dressing of claim 21, wherein the hydrophobic filter pieces have a size distribution such that at least about 80% by weight of the filter pieces have a major dimension in the range of from about 1.0 mm to about 2.0 mm and a minor dimension in the range of from about 1.0 to about 2.0 mm.

31. The dressing of claim 30, wherein the hydrophobic filter pieces comprise a milled foam made of polyurethane.

32. (canceled)

33. The dressing of claim 21, wherein the composition comprises an antimicrobial agent comprising silver.

34. (canceled)

35. The dressing of claim 21, wherein the permeable, non-adherent envelope comprises a nylon and at least a portion having a pore density in the range of from about 2 pores/cm2 to about 1,000 pores/cm2.

36. (canceled)

37. The dressing of claim 21, further comprising:
a cover configured to be placed over the secondary layer so as to seal to tissue surrounding the tissue site to form a sealed space.

38. The dressing of claim 37, wherein the hydrophobic filter pieces are configured to capture at least some of a patient's growth factors or enzymes released proximate to the dressing.

39. The dressing of claim 38, wherein the hydrophobic filter pieces are configured to release at least some of the growth factors or enzymes from the dressing, when the dressing is rinsed with saline.

40. A method for providing negative pressure therapy to a tissue site, the method comprising:
positioning a dressing comprising a composition comprising hydrophobic filter pieces, a permeable, a non-adherent envelope enclosing the composition, a secondary layer, and a sealing member such that the non-adherent envelope enclosing the composition is adjacent to the tissue site, the secondary layer is positioned over the non-adherent envelope enclosing the composition, and the sealing member is positioned over the secondary layer;
sealing the dressing to tissue surrounding the tissue site to form a sealed space;
fluidly coupling a negative-pressure source to the sealed space; and
operating the negative-pressure source to generate a negative pressure in the sealed space.

41. The method of claim 40, wherein the composition further comprises collagen.

42. (canceled)

43. The method of claim 41, wherein the composition further comprises oxidized regenerated cellulose (ORC).

44-53. (canceled)

54. The method of claim 40, wherein, upon operating the negative-pressure source to draw fluid from the sealed space through the non-adherent envelope enclosing the composition is effective to filter biological molecules from the fluid.

55. The method of claim 40, further comprising modulating enzyme activity, particularly, protease activity at the tissue site by capturing the enzymes, particularly, proteases within the non-adherent envelope enclosing the composition and removing the non-adherent envelope enclosing the composition with the captured enzymes, particularly, proteases.

56. The method of claim 40, further comprising delivering signaling molecules, particularly, growth factors, to the tissue site by capturing the signaling molecules, particularly, growth factors, within the non-adherent envelope enclosing the composition; removing the captured signaling molecules, particularly, growth factors, from the non-adherent envelope enclosing the composition; and returning the signaling molecules, particularly, growth factors to the tissue site.

57. A method for enhanced healing of a wound, the method comprising:
capturing growth factors or enzymes released from the wound in a dressing comprising a permeable, non-adherent envelope enclosing a composition comprising hydrophobic filter pieces; and
releasing at least some of the captured growth factors from the dressing to the wound.

58. The method of claim 57, wherein saline is applied to the dressing in order to release at least some of the captured growth factors or enzymes from the dressing.

59. The method of claim 58, wherein the composition further comprises collagen.

60. (canceled)

61. The method of claim 59, wherein the composition further comprises oxidized regenerated cellulose (ORC).

62-64. (canceled)

65. The method of claim 57, wherein the hydrophobic filter pieces have a size distribution such that at least about 80% by weight of the filter pieces have a major dimension in the range of from about 1.0 mm to about 2.0 mm and a minor dimension in the range of from about 1.0 to about 2.0 mm.

66. The method of claim 57, wherein the hydrophobic filter pieces comprise a milled foam made of polyurethane.

67. (canceled)

68. The method of claim 57, wherein the composition comprises an antimicrobial agent comprising silver.

69. (canceled)

70. The method of claim 57, wherein the permeable, non-adherent envelope comprises a nylon and at least a portion having a pore density in the range of from about 2 pores/cm2 to about 1000 pores/cm2.

71. (canceled)
